(19) United States
(12) Patent Application Publication
(10) Pub. No.: US 2009/0048498 A1
(43) Pub. Date: Feb. 19, 2009

(54) System and method of monitoring an animal

(76) Inventor: Frank Riskey, Boise, ID (US)

Correspondence Address: STOEL RIVES LLP - SLC, 201 SOUTH MAIN STREET, SUITE 1100, ONE UTAH CENTER, SALT LAKE CITY, UT, 84111, US

(21) Appl. No.: 11/893,928

(22) Filed: Aug. 17, 2007

(51) Int. Cl. A61B 1/00

(52) U.S. Cl. 600/302

(57) ABSTRACT

A system and method for monitoring an animal is disclosed. In particular, an ingestible bolus configured to be maintained in the stomach of an animal is disclosed where the bolus comprises a sensor to monitor physiological and other characteristics of the animal. The bolus comprises a data transmitter in wireless communication with a base station and/or transponder in communication with a base station. Within the animal stomach, the bolus may be generally disposed in either a first orientation or second orientation. The base station and/or transponder comprises a plurality of antennae each having an orientation corresponding to a likely orientation of the bolus within the animal in order to reduce the power requirements of the bolus and increase its operational range. The base station may be configured to receiving incoming signals on each of the antennae and may combine the signals into a single input signal.

### TECHNICAL FIELD

This invention relates to animal monitoring systems and methods, in particular, to systems and methods for detecting a health-condition of an animal using an ingestible bolus maintained within the body of an animal in wireless communication with a base station.

### BRIEF DESCRIPTION OF THE DRAWINGS

The various aspects and advantages of the invention are described by way of example in the following description of several embodiments and attached drawings. It should be understood that the accompanying drawings depict only typical embodiments and, as such, should not to be considered to limit the scope of the claims. The embodiments will be described and explained with specificity and detail in reference to the accompanying drawings in which:

FIG. 1 is a diagram of one embodiment of an animal monitoring system according to the teachings of the present invention;

FIG. 2 is a block diagram of one embodiment of a bolus according to the teachings of the present invention;

FIG. 3 is a diagram of two boluses disposed in two alternate orientations within a stomach of a ruminant animal; and

FIG. 4 is a flow diagram of a processing method for monitoring an animal according to the teachings of the present invention.

### DETAILED DESCRIPTION OF PREFERRED EMBODIMENTS

The present invention is directed to a system and method for monitoring physiological and other characteristics of animals in order to monitor and detect the health risks and condition of such animals.

It will be readily understood that the components of the present invention, as generally described and illustrated in the Figures herein, could be arranged and designed in a wide variety of different configurations. Thus, the following detailed description of the embodiments of the apparatus, system, and method of the disclosure is not intended to limit the scope of the disclosure, as claimed, but is merely representative of possible embodiments of the disclosure.

In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the Figures herein could be arranged and designed in a wide variety of different configurations.

The order of the steps or actions of the methods described in connection with the embodiments disclosed may be changed as would be apparent to those skilled in the art. Thus, any order in the Figures or description is for illustrative purposes only and is not meant to imply a required order, unless specified to require an order.

Certain aspects of the embodiments described may be illustrated as hardware components, or software modules or components. As used herein, a software module or component may include any type of computer instruction or computer executable code located within a memory device and/or transmitted as electronic signals over a system bus or wired or wireless network. A software module may, for instance, comprise one or more physical or logical blocks of computer instructions, which may be organized as a routine, program, object, component, data structure, etc., that performs one or more tasks or implements particular abstract data types. In certain embodiments, a particular software module may comprise disparate instructions stored in different locations of a memory device, which together implement the described functionality of the module. Indeed, a module may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules may be located in local and/or remote memory storage devices.

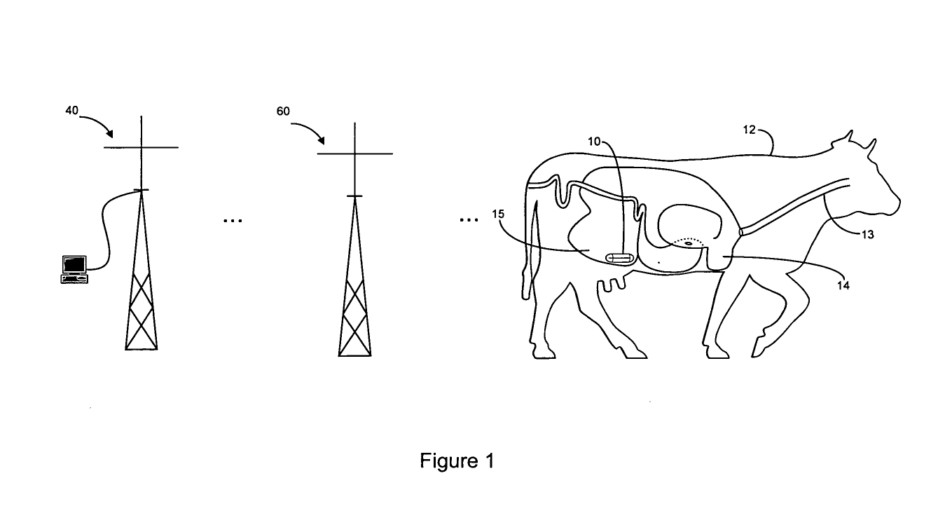

Turning to FIG. 1, one embodiment of a bolus 10 may be disposed within the body of an animal 12. In this embodiment, bolus 10 may be configured to be ingested via the esophagus 13 of a ruminant animal 12, such as a bovine. In this embodiment, bolus 10 may be configured to have a size and density which will enable it to remain within the stomach of bovine 12, ensuring that it is not regurgitated from the animal's rumen 15 or reticulum 14. Bolus 10 may be capable of remaining in the animal's rumen 15 or reticulum 14 throughout the life of animal 12. In an alternative embodiment, bolus 10 may be injected under the skin of an animal or otherwise implanted within the body of an animal 12.

Bolus 10 may comprise wireless communications means and be in wireless communication with base station 40. In some embodiments, such wireless communication may be two-way, allowing bolus 10 to both transmit to and receive data from base station 40. In other embodiments, bolus 10 may only be capable of transmitting data to base station 40.

In some embodiments, animal 12 may be capable of roaming large distances. As such, the distance between base station 40 and bolus 10 may become greater than the wireless transmission range of bolus 10. In this case, wireless transponder 60 may be deployed to increase the communications range of bolus 10 to base station 40. In this embodiment, transponder 60 may receive wireless transmissions from bolus 10 and retransmit them at a higher power and/or different frequency to allow such transmissions to be received by base station 40. Similarly, in this embodiment, transponder 60 may receive transmissions from base station 40 to bolus 10 and retransmit them at higher power so that they may be received by bolus 10.

In the embodiment of FIG. 1, bolus 10 may comprise one or more sensors to detect one or more physiological or other characteristics of animal 12. In this embodiment, bolus 10 may wirelessly transmit data corresponding to monitored animal characteristics to base station 40. Such animal characteristics may include physiological characteristics, such as animal temperature, stomach pH, blood pH, heart rate, respiration, stomach or rumen contractions, and the like. Such characteristics may also include non-physiological characteristics, such as animal movement or animal position.

In the embodiment of FIG. 1, base station 40 may receive messages comprising animal characteristics transmitted from bolus 10. Base station 40 may comprise software configured to execute a software-implemented process to monitor animal 12. In this embodiment, base station 40 may forward messages received from bolus 10 to the process configured to monitor animal 12. The animal monitoring process may determine whether animal's health is at risk or whether animal 12 is experiencing a change in its health condition. Such detection may comprise comparing the animal measurements received from bolus 10 to a set of base-line characteristics comprising an animal profile. As used herein, an animal profile may refer to stored characteristics corresponding to a particular animal 12. Alternatively, profile as used herein may refer to stored characteristics corresponding to a particular breed and/or sex of animal (e.g., a profile corresponding to Holstein dairy cows), or may refer to a set of stored characteristics corresponding to a particular set or group of animals.

In one embodiment, any health risks or changes in health condition detected by the animal monitoring process may be stored as a profile associated with the animal 12. Such a profile may comprise a relational or object-oriented database record, an entry in a file-system, an entry in a data file, or any other data storage or management technique known in the art. The animal monitoring process running in conjunction with base station 40 may consult the animal specific profile in order to more accurately detect health risks to animal 12 and/or changes in the health condition of animal 12. For example, if the monitoring process running in conjunction with base station 40 were to detect that animal 12 was in an estrus state, this state may be recorded in the profile associated with animal 12. Then, upon receipt of subsequent message from animal 12, the monitoring process may monitor for estrus-specific conditions and/or for a change to a non-estrus state in animal 12. Additionally, the animal specific profile may allow an animal manager to query the animal monitoring process of base station 40 to obtain the current health risks and health condition of a particular animal 12.

In one embodiment, the animal monitoring process running in conjunction with base station 40 may alert an animal manager in the event that a risk to the health of animal 12 or a change in the health condition of animal 12 is detected. As used herein, an animal manager may refer to a human or other entity capable of managing an animal, including, but not limited to, responding to the health risks of an animal 12, responding to a health condition of an animal 12 (e.g., animal in estrus state or calving), responding to a change in location of an animal 12 (e.g., whether the animal is outside of its enclosure), or otherwise managing the animal (e.g., providing food, dietary supplements, modifying environmental conditions, etc).

In this embodiment, base station 40, and the animal monitoring process running in conjunction with base station 40, may be communicatively coupled to a communications network including, but not limited to: a local area network (LAN), the Internet, a cellular telephone network, a telephone network, such as a Public Switched Telephone Network (PSTN), or the like. In this case, the animal monitoring process may alert an animal manager of a health risk to animal 12 or a change in health condition to animal 12 via one or more of these communications networks, allowing the animal manager to appropriately respond to the situation in a timely manner.

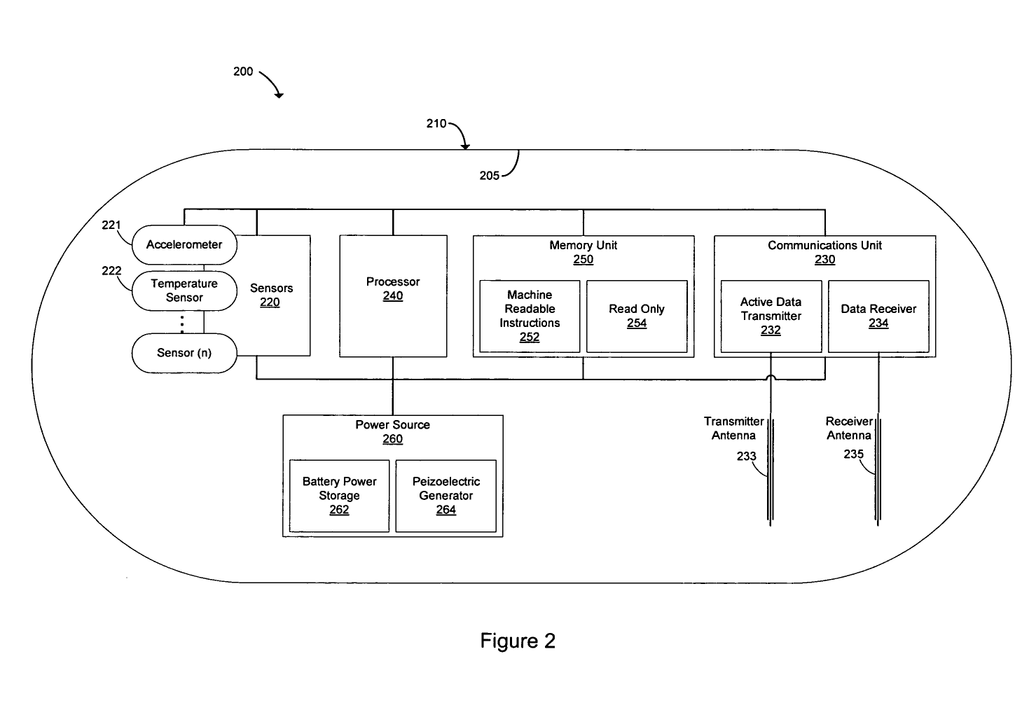

Turning now to FIG. 2, a block diagram 200 is shown of one embodiment of a bolus 210. The components of bolus 210 may be disposed within an enclosure 205. Enclosure 205 may be formed from any material capable of remaining within the stomach of an animal without deteriorating or degrading. In one embodiment, enclosure 210 is formed from a plastic material.

Bolus 210 may comprise one or more sensors 220 to measure animal characteristics. One or more of sensors 220 may detect animal movement characteristics including, but not limited to: distance traveled by the animal, animal movement frequency, animal movement speed, and the like. In one embodiment, an accelerometer 221 may be used to detect such movement characteristics. In this embodiment, accelerometer 221 may be a three (3) axis accelerometer capable of detecting animal movement in each of the Cartesian “x,” “y,” and “z” axes. Detecting movement in each of these three axes may be important since bolus 210 may change its orientation while within the stomach of an animal. As such, detection of movement in only one or two axes may yield inaccurate results.

An acceleration vector magnitude (VM) value may be calculated from the readings of the 3-axis accelerometer by calculating the square root of the sum of the squares of each of the “x,” “y,” and “z” coordinate axes as illustrated by equation 1.1:

VM=\sqrt{x²+y²+z²} Eq. 1.1

A derivative of the vector magnitude (VM) may be approximated by calculating the absolute value of the difference between subsequent vector magnitude values as illustrated in equation 1.2.

$$\frac{\partial {VM}_{n}}{\partial t} = \vert {VM}_{n} - {VM}_{n - 1} \vert \qquad Eq . 1.2$$

The derivative of acceleration calculated per equation 1.2 may be useful in monitoring animal characteristics as it may remove sensor areas caused by “float” movement of bolus 210 within the stomach of the animal or other constant acceleration forces acting on the animal 12 (e.g., gravity). Accordingly, the derivative of the movement vector magnitude may provide a more accurate representation of the actual movement characteristics of the animal. Additionally, the derivative value approximated by equation 1.2, may be indicative of how “erratic” the movement of animal 12 is; a large acceleration derivative value may indicate significant starting and stopping of movement in animal 12.

In one embodiment, bolus 210 may comprise one or more sensors 220 capable of determining the position of bolus 210, such as a Global Positioning System (GPS) receiver. A GPS receiver may be used to detect both animal position and animal movement characteristics.

One or more sensors 220 of bolus 210 may be used to detect internal physiological characteristics of an animal including, but not limited to: body temperature, heart rate, respiration, stomach contractions, stomach pH, blood pH, and the like. Any number of sensors 220 may be used to detect such characteristics. For example, to detect animal temperature, a temperature sensor 222 may be employed. In this embodiment, temperature sensor 222 may comprise a thermistor, thermocouples or a platinum resistance thermometer or the like.

It would be understood by one skilled in the sensor arts that any number of sensors 220 could be included within bolus 210 under the teachings presented herein. As such, this disclosure should not be construed as limited to any particular sensors 220.

In one embodiment, bolus 210 may comprise a communications unit 230. Communications unit 230 may comprise active data transmitter 232 and data receiver 234. Active data transmitter 232 may be communicatively coupled to transmitter antenna 233. Transmitter antenna 233 may be disposed within enclosure 205 of bolus 210, upon the surface thereof, or may be disposed externally to enclosure 205 of bolus 210. Data receiver 234 may be communicatively coupled to receiver antenna 235. Receiver antenna 235 may be disposed within enclosure 210 of bolus 10, upon the surface thereof, or may be disposed externally to enclosure 210 of bolus 10. In one embodiment, transmission antenna 233 may be capable of transmitting data at 900 MHz, and receiving antenna 235 may be capable of receiving data at 900 MHz. In another embodiment, transmission antenna 233 and receiving antenna 235 may be comprised of a single antenna (not shown) used for both data transmission and reception.

Bolus 210 may comprise a processor 240 communicatively coupled to a memory unit 250. In one embodiment, memory unit 250 may comprise machine readable instructions 252 stored thereon. In this embodiment, processor 240 may read and execute machine readable instructions 252 stored on memory unit 250.

Processor 240 may be communicatively coupled to each of sensors 220. Machine readable instructions 252 stored on memory unit 250 may specify a sensor sampling frequency for each of the sensors 220. As used herein, a sensor sampling frequency may determine how often a sensor reading is obtained from a particular sensor 220. For example, a sensor sampling frequency may define how often temperature sensor 222 obtains a temperature sensor reading or sensor sample from the animal. The processor 240 may configure one or more of sensors 220 with a sensor sampling frequency specified by machine readable instructions 252. Alternatively, one or more sensors 220 may be communicatively coupled to memory unit 250 and may be configured to read their sensor sampling frequency directly from the machine readable instructions 252.

Machine readable instructions 252 may specify a sensor reading duration for each of sensors 220. As used herein, a sensor reading duration may define the length of time a particular sensor 220 may obtain a reading. For example, a reading duration may define how long accelerometer 221 reads animal movement characteristics. A reading duration may specify that accelerometer 221 should read animal movement characteristics for one minute each time a sensor sample is taken. Processor 240 may configure one or more of sensors 220 with a sensor reading duration specified by machine readable instructions 252. Alternatively, one or more sensors 220 may be communicatively coupled to memory unit 250 and may be configured to read their sensor reading duration directly from machine-readable instructions 252.

Machine readable instructions 252 may specify calibration information for one or more sensors 220. In this embodiment, one or more sensors 220 may be tested to determine whether it is returning accurate readings. In the event a particular sensor 220 is not returning accurate readings, calibration data may be stored within memory unit 250 to rectify the readings to a correct value. In this embodiment, sensor 220 may be communicatively coupled to memory unit 250 to allow a sensor 220 to read the calibration data therefrom. Sensor 220 may itself comprise a memory storage location whereon such calibration information may be stored. Machine readable instructions 252 may instruct processor 240 to transfer sensor calibration data stored within memory unit 250 to the memory storage location of a particular sensor 220. In another embodiment, sensor 220 may not comprise a memory storage location and may not be capable of reading memory unit 250. As such, machine readable instructions 252 may configure processor 240 to apply calibration data stored within memory unit 250 to readings returned by sensors 220.

Machine readable instructions 252 may specify that one or more sensors 220 should be deactivated in order to reduce the power consumed by bolus 210. Processor 240 may be communicatively coupled to sensors 220 and may be capable of configuring and/or controlling one or more of sensors 220. Machine readable instructions 252 may specify that one or more sensors 220 should be re-activated.

Processor 240 may be communicatively coupled to sensors 220 and may control the operation and configuration of sensors 220. Processor 240 may poll one or more of sensors 220 at a polling interval specified by machine readable instructions 252 stored in memory unit 250. As used herein, polling a sensor refers to obtaining measurement data from one or more sensor 220. Polling a sensor may comprise processor 240 sending a query to a sensor 220, and, responsive to this query, sensor 220 may obtain and return to processor 240 the sensor reading. For example, temperature sensor 222 may respond to polling by reading and returning the current animal temperature. In another embodiment, polling a sensor may simply comprise processor 240 reading the current sensor value from a sensor. In another embodiment, one or more sensors 220 may be configured to store sensor measurements on memory unit 250. One or more sensors 220 may be configured with a sensor sampling frequency that is greater than the polling frequency of processor 240. As such, sensors 220 may store multiple sensor samplings on memory unit 250 between polling intervals of processor 240. Accordingly, polling a sensor 220 may comprise processor 240 reading all of the sensor readings stored on memory unit 250 for each of the one or more sensors 220.

In another embodiment, sensor 220 may alternatively comprise a memory storage location to store sensor samples. In this embodiment, processor 240 may poll sensor 220 by reading a sensor 220 storage location. In another embodiment, sensor 220 may have a sensor reading duration to allow sensor 220 to measure animal characteristics over time (e.g., an accelerometer sensor 221). Sensor 220 may store such measurements on an internal sensor storage location or on memory unit 250. The processor 240 may poll such a sensor by reading memory 250 or the internal storage location of the sensor 220.

It should be understood that bolus 210 may comprise sensors 220 having any number of sampling or measurement storage techniques and that processor 240 may be configured by machine readable instructions 252 to poll sensors 220 having such various sampling or measurement storage techniques.

Machine readable instructions 252 may specify a polling frequency for each sensor 220 or may specify a common polling internal all or a sub-set of sensors 220. As used herein, a polling frequency may specify how often processor 240 polls one or more sensors 220.

In one embodiment, machine readable instructions 252 may define conditions under which the polling frequency associated with one or more sensors 220 may change. For example, machine readable instructions 252 may instruct processor 240 to increase the polling frequency and/or sensor sampling frequency of a temperature sensor 222 in the event that the animal temperature exceeds a threshold value. Instructions 252 may instruct processor 240 to decrease the polling frequency and/or sensor sampling frequency of the temperature sensor 222 if the animal temperate is maintained below the threshold value. Processor 240 may adapt the polling frequency and/or sensor sampling frequency to changing animal health conditions so that potential health risks and/or other changes in animal health state may be recognized as soon as possible while minimizing extraneous sensor measurements and message transmissions.

In one embodiment, processor 240 may transmit sensor measurements obtained by polling sensors 220 via data transmitter 232. In one mode of operation, processor 240 may form a message comprising the measurements as sensor 220 readings are obtained (after polling the one or more sensors 220). Such a message may be referred to as an animal characteristics message, and may be comprised of the sensor readings obtained by polling one or more sensors 220. This operational mode may be referred to as “instantaneous” mode since sensor readings are transmitted as they are polled by processor 240. In another mode of operation, processor 240 may not immediately transmit the sensor readings polled from sensors 220, but instead store them on memory unit 250. In this mode, machine readable instructions 252 may specify a transmission internal, wherein processor 240 may transmit an animal characteristics message comprising some or all of the measurements stored in memory unit 250 at each transmission interval. This operational mode may be referred to as “burst” mode since sensor 220 readings are transmitted as periodic bursts rather than when sensor polling takes place. Operation in “burst” mode may reduce the power consumed by bolus 210 by reducing the number of transmissions sent from data transmitter 232.

In one embodiment, messages transmitted via data transmitter 232 of communications unit 230 may comprise a media access control (MAC) value. A MAC may be a 6 byte value used to uniquely identify messages originating from a particular bolus 210. A MAC value may also be used by data receiver 232 and/or processor 240 to identify messages intended for bolus 210. As such, receiver 232 and/or processor 240 may disregard any incoming messages having a MAC address than its own, obviating the need to time-slice or otherwise manage wireless traffic between bolus 210 and a base station or other wireless device. MAC addressing to route and control network messages is generally known within the networking arts.

In one embodiment, a programmable unique animal identifier (UAID) may be stored on memory unit 250. In this embodiment, the UAID may be used to associate a bolus 210 with a particular animal. The UAID value may be transmitted with some or all of the messages originating from a particular bolus 210, allowing the receiver of such messages to associate the received data with a particular animal.

In one embodiment, the bolus memory may comprise read-only storage 254. Read-only storage 254 may be a Programmable Read-Only Memory (PROM), Erasable Programmable Read-Only Memory (EPROM), Electrically Erasable Programmable Read-Only Memory (EEPROM), or the like. In this embodiment, a unique bolus identifier value (UBID) may be stored within the read-only storage 254. The UBID value may be transmitted with some of all of the messages transmitted from the bolus 210. In this embodiment, the UBID may provide a tamper-proof identifier to uniquely identify a particular bolus 210.

In one embodiment, communications unit 230 may detect whether bolus 210 is within range of a receiver, such as a base station (not shown) or transceiver (not shown). Processor 240 may cause communications unit 230 to transmit a simple message at a set interval. This simple message may be referred to as a “ping” and may include one or more of the unique identifiers associated with a particular bolus 10 (e.g., a MAC, UAID, and/or UBID). A base station or transceiver receiving the ping message may be configured to send a short reply message indicating that the ping message was received. In this way, processor 240 may know that it is within wireless range of a base station or transceiver. Upon receipt of a reply message, bolus 210 may be configured to be in “on-line” mode. If bolus 210 does not receive a reply message within a threshold period of time, it may transmit additional ping messages. If a threshold number of retry ping messages have been sent without a reply, bolus 210 may be configured to be in “off-line” mode. Machine readable instructions 252 may include instructions to be executed by processor 240 corresponding to “on-line” and/or “off-line” mode.

In “on-line” mode, bolus 210 may transmit animal characteristics messages at the “online” transmission frequency specified by machine readable instructions 252. As discussed above, such messages may be transmitted as processor 240 polls sensors 220, or may be transmitted at a periodic transmission interval. The receiver of such messages may be configured to respond with a confirmation message. The confirmation message may be used in the place of a separate ping message in order to decrease the message traffic between bolus 210 and the receiver.

In “off-line” mode, bolus 210 may decrease transmission frequency of messages according to machine readable instructions 252. Additionally, while in “off-line” mode, machine readable instructions 252 may direct processor 240 to deactivate certain sensors 220 in order to conserve power. In “off-line” mode, bolus 10 may continue sending “ping” messages in order to discover when bolus 210 comes back into range of a base station or transceiver unit. In this sense, data transmitter 234 of bolus 210 may be considered to be an active transmitter since bolus 210 may actively transmit animal characteristic messages and may actively detect when a base station or transceiver is in wireless communications range. Bolus 210 may actively transmit animal characteristics and/or detect wireless communications without requiring interrogation by an external source.

In one embodiment, bolus 210 may receive new and/or modified machine readable instructions 252 via data receiver 234 of wireless communications unit 230. Such received instructions may comprise changes to the operation of sensors 220 and/or processor 240 including: sensor sampling frequency; sensor reading duration; sensor activation status; sensor calibration data; processor polling frequency; processor operational mode (i.e., “instantaneous” or “burst); and the like.

The embodiment of FIG. 2 may comprise power source 260 coupled to each of sensors 220, communications unit 230 including data transmitter 232 and data receiver 234, processor 240, memory unit 250, and any other power consuming component of bolus 210. Power source 260 may comprise a battery energy storage device 262, such as a lithium ion battery, lead acid battery, nickel cadmium battery, or the like. In another embodiment, power source 260 may comprise a generator 264. In one embodiment, generator 264 may be a piezoelectric generator or mass/alternator generator to generate power from the movement or vibration of bolus 210 within a host animal. In another embodiment, generator 264 may be a heat-activated generator to generate electrical energy from the body heat of a host animal. In some embodiments, generator 264 may be disposed outside of the bolus 10 enclosure. Power source 260 may comprise both battery power storage 262 and generator 264; in this embodiment, power generated by power generator 264 may be stored in battery power storage 262.

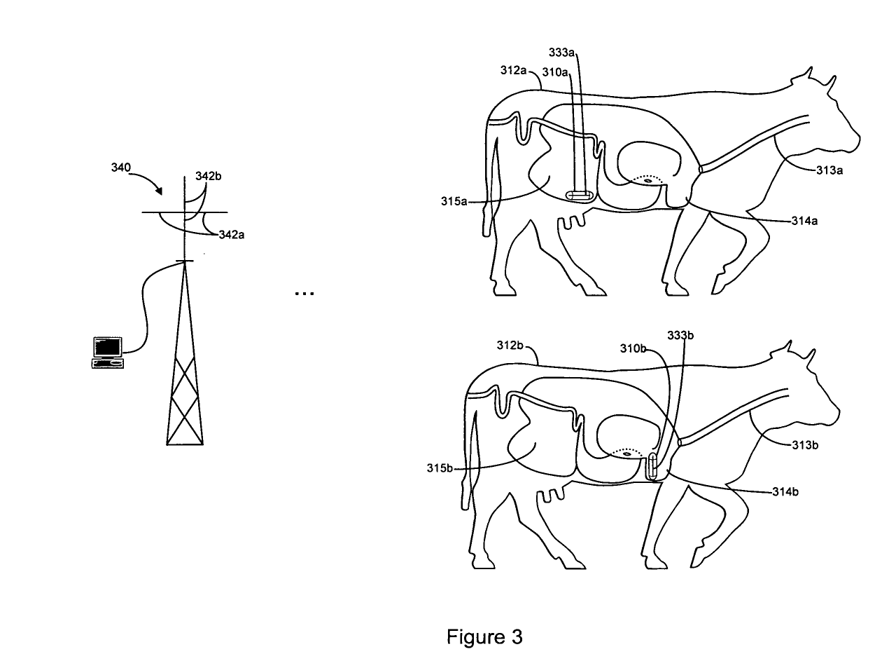

Turning now to FIG. 3, illustrating boluses 310a and 310b disposed within animals 312a and 312b, respectively. Boluses 310a and 310b each comprise transmitter antennae 333a and 333b. Boluses 310a and 310b may be adapted to be ingested by a ruminant animal 312a, 312b and maintained within the animal's rumen 315a, 315b or reticulum 314a, 314b for potentially the life of the animal 312a, 312b. It has been observed that while within the animal rumen 315a, 315b or reticulum 314a, 314b, boluses 310a and 310b may be maintained in one of two possible orientations. FIG. 3 shows a bolus 310a in a first orientation, and bolus 310b is depicted in a second orientation. The orientation of bolus 310a may be substantially orthogonal to the orientation of bolus 310b. Similarly, the orientation of antenna 333a of bolus 310a may be substantially orthogonal to the orientation of antenna 333b of bolus 310b. It has been observed that the orientation of bolus 310a may be substantially horizontal with respect to animal 312a, and that the orientation of bolus 310b may be substantially vertical with respect to animal 312b. Accordingly, the orientation of bolus 310a may be substantially orthogonal to the orientation of bolus 310b, and the orientation of antenna 333a may be substantially orthogonal to the orientation of antenna 333b. As such, the radio frequency (RF) wave-form generated by antenna 333a of bolus 310a may be substantially orthogonal to the orientation of the signal generated by antenna 333b of bolus 310b.

The difference in orientation between antennae 333a and 333b may decrease the operational communications range of bolus 310a and 310b. Since the wave-forms generated by antennae 333a and 333b are substantially orthogonal, a receiving antenna may not be capable of efficiently receiving signals from one or the other orientation. In most wireless environments, wireless signals are most efficiently received when the transmitting antenna is substantially aligned with the receiving antenna. As such, if a receiving antenna were to be substantially aligned with antenna 333a, bolus 310a would be capable of efficiently communicating over relatively long distances using relatively low power. However, antenna 333b would be oriented substantially orthogonally to the antenna, significantly decreasing the range of bolus 310b. Further, since boluses 310a and 310b may shift between the first orientation (shown by 310a) and second orientation (shown by 310b), while within the animal 312a, 312b, it may be difficult to predict which receiving antenna orientation to select for a given bolus or determine the true operational range of a boluses 310a, 310b. Moreover, in environments having more than one bolus in operation, it is highly unlikely that all the boluses 310a, 310b will have the same orientation at any given time. Furthermore, if an antenna were oriented at an angle between first bolus orientation 310a and second bolus orientation 310b (i.e., substantially 45° relative to 333a and 333b), both boluses 310a and 310b would have a similar range, but that range would not be maximal nor would it maximize power efficiency.

Base station 340 may be in wireless communication with boluses 310a and 310b regardless of the orientation of boluses 310a, 310b. Base station 340 may be comprised of two receiver antennae; antenna 342a may have a first orientation and 342b may have a second orientation. The orientation of first receiver antenna 342a may correspond to first bolus orientation 310a, and the orientation of second receiver antenna 342b may correspond to second bolus orientation 310b. Base station 340 may be configured to add the RF signals received by antennas 342a and 342b to generate a single received signal. As such, base station 340 may efficiently receive signals from boluses 310a and 310b regardless of the orientation of boluses 310a and 310b. It should be noted that additional antennae could be added to base station 360 depending upon the observed orientation characteristics of boluses 310a, 310b within a given animal.

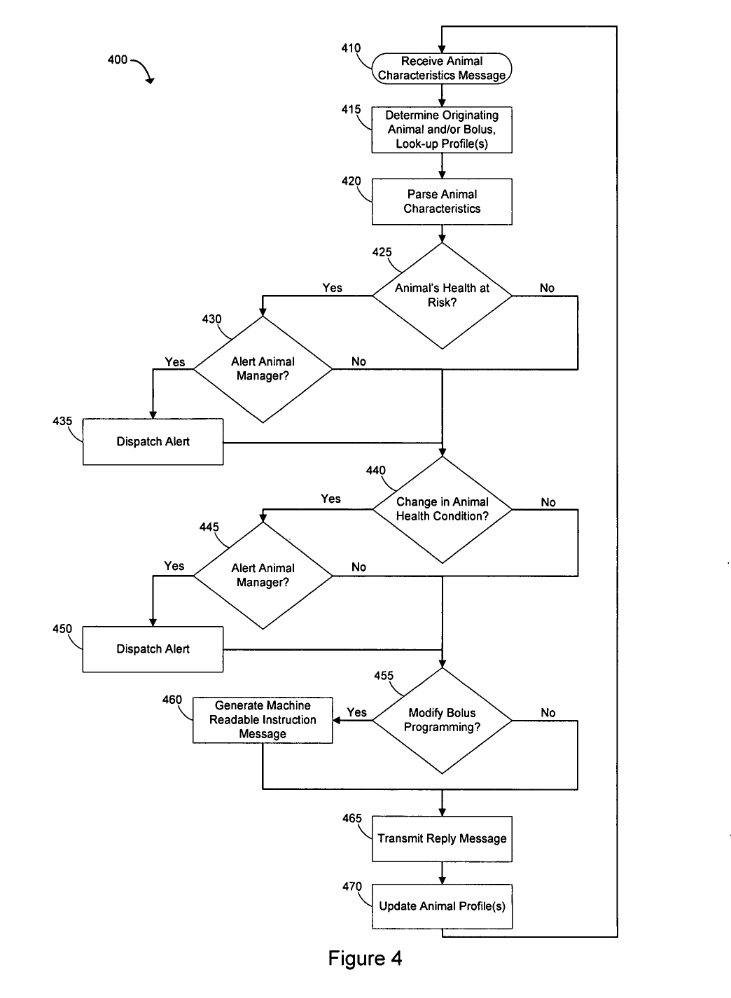

Turning now to FIG. 4, a process flow diagram 400 is shown comprising steps that may be executed on a base station in wireless communication with a bolus. In this embodiment, a base station may comprise a computing device, such as a personal computer running an operating system, such as Linux or Microsoft® Windows. Accordingly, the steps of the flow diagram 400 may be executed by a software program embodied as machine readable instructions running on the computing device. The software program may be communicatively coupled to a wireless communications system of a base station allowing the software program to send and receive messages from bolus devices within its wireless communications range.

At 410 the process may receive an animal characteristics message from a bolus disposed within an animal to be monitored by process 400. The animal characteristics message may be received wirelessly by one or more antennae communicatively coupled to a base station. Process 400 may be configured to receive all messages received by the base station.

At 415, the process may parse the animal characteristics message received at step 410 to determine the source animal and/or bolus. In one embodiment, the process may perform this step by reading a media access control (MAC) value from the animal characteristics message and using the MAC value as an input into a look-up table or relational database associating MAC addresses to particular animals and/or boluses. Alternatively, or in addition, to a MAC value, the animal characteristics message may comprise a unique animal identifier (UAID) or a unique bolus identifier (UBID) that the process may use to determine the originating bolus and/or animal.

Determining the source bolus and/or animal at step 415 may further comprise accessing one or more profiles associated with the bolus and/or animal. Such profiles may include a profile associated with a particular animal, group of animals, breed/sex of animals, or the like. For example, at 415 the process may obtain a profile associated with a particular animal and a profile associated with the animal's breed (i.e., a Holstein dairy cow).

The animal profile information accessed at step 415 may comprise data associated with the animal or bolus. Such information may comprise the current state of the source bolus, such as the current level of charge within the bolus power source, the polling frequency of each of the bolus sensors, base-line characteristics of the animal, and the like. Animal profile information accessed at step 415 may comprise animal characteristics data. For instance, a profile associated with a particular breed/sex of animal may comprise general threshold parameters, such as nominal animal temperature, movement activity, and the like. Similarly, an animal profile associated with a particular animal may comprise past characteristic data received from the animal including, the current health condition of the animal (i.e., whether the animal is currently in a estrus state), any animal-specific information (i.e., animal tends to exhibit more movement activity than others), and the like.

At 420, the process may access animal characteristics comprising the animal characteristics message received at 410. As discussed above, such animal characteristics may comprise measurements corresponding to the internal physiological state of the animal (e.g., temperature, rumen pH, etc) and/or measurements corresponding to other animal characteristics (e.g., animal movement, animal position, etc.). The animal characteristics contained within the message received at 410 may comprise the instantaneous readings of one or more bolus sensors if the source bolus is operating in “instantaneous” mode or may comprise a series of readings if the source bolus is operating in “burst” mode.

At 425, the process may assess the animal characteristics obtained at step 420 to determine whether the animal's health is at risk. This determination may be made by comparing the animal characteristics obtained at step 420 to the animal profile(s) accessed at 415. The animal profile data accessed at 415 may comprise data common to all animals of a particular breed or type (e.g., Holstein dairy cows), be specific to the particular animal, and/or may correspond to a group of animals. These animal profiles may define one or more health risk conditions. For example, one such health risk condition could be a “high-temperature” condition where an animal health risk is registered if the animal temperate exceeds a threshold value. Such a threshold value may be defined in a profile common to all animals of a particular breed, or may be defined on a per-animal basis. In addition, the determination of step 425 may comprise comparing the sensor readings obtained at 420 to past sensor readings. For example, an animal health risk may be triggered if the bolus movement sensor has not registered any animal movement for some threshold time period as this may indicate that the animal has become immobilized or is otherwise incapacitated. Similarly, an operator may define non-health conditions that may trigger an animal health risk at 425. If the animal characteristics obtained at 420 were to comprise animal position information (e.g., a GPS reading), a health risk event could be triggered if the animal were to be outside of a defined range or enclosure area. Such a condition may be defined in a profile associated with a particular group of animals where the group is known to be housed in a particular enclosure (i.e., all the animals are in the same pasture or feed lot). If the determination of 425 indicates a potential health risk to the animal, the flow may continue to 430. Otherwise, the flow may continue to 440.

At 430, the program may determine whether the health risk identified at 425 poses an immediate danger to the animal and, as such, requires immediate attention from an animal manager. As in step 425, the animal profile(s) accessed at step 415 may define whether a particular health risk requires an alert at 435. For example, an animal profile may indicate a temperature health risk at 425 if the animal's temperature exceeds a threshold value (i.e., animal is three degrees above normal). Additionally, the profile may indicate an immediate health risk to the animal warranting an alert at step 435 if the animal temperature further exceeds the threshold value (i.e., six degrees above normal) or has been maintained above normal for some period of time (i.e., animal is three degrees above normal for two days). If the determining at step 430 indicates that the health risk to the animal warrants an alert, process 400 may continue to 435, otherwise process 400 may continue to 440.

At 435, the program may issue a health alert message to alert an animal manager of a health risk facing the animal. Embodiments of the present invention may issue such an alert in any number of ways. In some embodiments, the process may include communicating with a local area network (LAN) and/or the Internet. In these embodiments, the process may cause a network message to be sent indicating that an animal needs immediate attention. Such a message may comprise an email, instant message, short message service (SMS), or the like. In some embodiments, the process may be communicatively coupled to a telephone or cellular telephone network. In these embodiments, the program may dispatch an alert message via voice, text, email, SMS, or the like. In other embodiments, the program may be communicatively coupled to an I/O system of a computing system comprising an audio speaker system. In these embodiments, the alert may comprise audible alert. In other embodiments, the I/O system may comprise a graphical user interface (GUI). In these embodiments, the program may display an alert message on the GUI. It should be understood that any combination alerting mechanisms known in the art could be used within the disclosed teachings and, as such, the disclosure should not be limited to any one or particular combination of alerting mechanisms. After dispatching the appropriate alert, process 400 may continue to step 440.

At step 440, the process may determine whether the animal characteristics obtained at step 420 correspond to a recognizable animal condition (e.g., an estrus state) and/or whether the animal is experiencing a physiological change. Such a physiological change could comprise a female entering an estrus cycle, ending an estrus cycle, calving, and the like. In one embodiment, detecting such a change may comprise comparing the received animal characteristics against a known animal health condition profile. Such a health condition profile may correspond to a particular breed and/or sex of an animal (e.g., Holstein dairy cows), or the health condition profile may correspond to a particular animal. For instance, a health condition profile for a Holstein dairy cow may specify that a one degree rise in animal body temperature is indicative of the beginning of a estrus cycle and a subsequent one degree drop in body temperature is indicative that the estrus cycle has ended. In this embodiment, if the animal characteristics obtained at 420 correspond to this profile, step 440 may detect a change in estrus state in the animal. Under the teachings of the present invention, any number of health condition profiles may be created corresponding to conditions including, but not limited to: estrus state, birthing/calving, impregnation, lactation, and the like. If the process at step 440 detects a health condition in the animal or a change in the health condition of the animal, the flow may continue to step 445, otherwise the flow may continue to step 455.

At 445, the process may determine whether the health condition identified at step 440 requires the attention of an animal manager. For example, if the determination at step 440 indicates that the animal is entering an estrus state, an animal manager may be notified in order to move the animal to a breeding area. Likewise, if the determination at step 440 indicates that an animal that was previously in an estrus state is no longer in this state or is impregnated, an animal manager may be notified in order to remove the animal from the breeding area. As in step 430, the animal profile(s) obtained at step 415 may define whether a particular animal condition warrants an alert. Such conditions may be established for a particular animal and/or for all animals within a group or breed. If the determination of step 445 indicates that an alert is required, the flow may continue at 450, otherwise the flow may continue at 455.

At 450, the program may issue an alert to an animal manager corresponding to the health condition or change in health condition of the animal. As discussed above in conjunction with step 435, the alert may be issued using any number of messaging techniques including, but not limited to: local area network communication, such as email, text, and SMS messages; cellular or public switched telephone network (PSTN) communication, such as voice, text, and SMS messages; or computer I/O, such as computer speakers, a GUI, or any other mechanism capable of alerting an animal manager to the animal's condition. After issuing an alert, the flow may continue to step 455.

At step 455, the process determines whether to change programming of the source bolus. Such change in program may comprise changes to: the activation status of one or more bolus sensors, the sensor sampling frequency of one or more bolus sensors, the sample duration of one or more bolus sensors, the polling frequency, the transmission interval, the operational mode of the bolus (e.g., “instantaneous” versus “burst” mode), and the like.

In some cases, the animal characteristics obtained at step 420 may deviate from the animal profiles obtained at step 415, but the changes may not rise to the level of representing a health risk per the determination of step 425 or a change in health condition per the determination of step 440. However, the deviation may indicate that a health risk or change in health condition may be forthcoming. As such, it may be desirable to increase the sensor sampling frequency, polling frequency, and/or transmission rate of the bolus in order to detect and respond to a possible change more quickly. For example, if the animal measurements received at 420 indicate that the animal may be entering an estrus cycle, the polling frequency of certain sensors within the bolus may be increased in order to more closely monitor the animal. This may be desirable since the estrus cycle of the animal may be relatively short, and early detection may increase the chances of successfully impregnating the animal. Similarly, it may be desirable to increase monitoring during the cycle in order to determine when the estrus cycle ends. Such detection may be important since the health risks to the animal may increase during its estrus cycle. During its estrus cycle, the animal may be placed in a breeding area in proximity to a breeding bull. This proximity may create a potential health risk for the animal. As such, an animal manager may wish to monitor the animal more frequency during estrus in order to detect completion of the animal's estrus cycle and/or impregnation as soon as possible to allow the animal to be removed from the potentially hazardous breeding area.

At step 455, the process may also determine whether the bolus sensor sampling frequency and/or polling frequency should be decreased and/or whether the transmission interval of the bolus should be increased. Such a change may be desirable if the animal characteristics obtained at 420 indicate that the animal is in a nominal health condition and close monitoring is not required. For instance, an animal that previously was closely monitored due to entering its estrus state, may no longer require such close monitoring once its estrus cycle has completed. Accordingly, at step 455, the process may decrease the monitoring frequency of the bolus once the animal characteristics have returned to normal.

If the determination at 455 indicates that a change to bolus programming should be made, process 400 may continue at step 460, otherwise process 400 may continue at 465.

At step 460, the process may generate updates and/or modifications to the machine readable instructions executed by the bolus to modify the bolus' operation per the determination of step 455. After the updates and/or modifications to the machine readable instructions have been generated, process 400 may continue at step 465.

At step 465, the process may transmit a message to the bolus. This message may comprise the modifications and/or updates generated at step 460. Alternatively, if the determining of step 455 indicated that no changes to bolus configuration was required and 460 was not performed, the message transmitted at step 465 may comprise a simple “acknowledge” message to confirm to the transmitting bolus that its message was received, obviating the need for the bolus to transmit a separate “ping.”

In one embodiment, the message transmitted at step 465 may comprise a MAC value to allow the message to be routed and identified by the intended recipient. Additionally, the message may include a UBID and/or UAID to further aid the bolus in identifying the message. After transmitting the bolus return message, the flow may continue at step 470.

At 470, the process may update one or more of the animal profiles retrieved at step 415. If an animal specific profile was obtained at step 415, the update of step 470 may comprise recording the animal characteristics received at 420 in the profile. Additionally, the update may comprise recording the health risk determination of step 425 and/or the health condition determination of 440. Such information may be used in subsequent iterations of the process in determining whether the health of the animal is deteriorating and/or whether the health condition of the animal is changing. Additionally, the animal profile may be compared against observed animal health risks and/or health conditions in order to refine the determination of steps 425 and 440. Upon the completion of step 470, the control flow of the process may return to step 410 where the system may wait for the receipt of animal characteristics message.

It should be understood that the flow described herein need not be executed in any particular order or be implemented by any particular technology. For example, the health risk determination of step 425 could be performed concurrently with the health condition determination of step 440 or, alternatively, the ordering of these steps could be reversed under the teachings of the present invention. Similarly, the alert of step 435 could be sent via a local area network connection, public switched telephone network (PSTN), or a personal computer input/output system. As such, the present invention should not be considered as tied to any particular implementation technology or any particular ordering of steps.

It will be obvious to those having skill in the art that many changes may be made to the details of the above-described embodiments without departing from the underlying principles of the invention. The scope of the present invention should, therefore, be determined only by the following claims.

1. An apparatus to monitor the health of an animal, comprising:
a bolus configured to be disposed within a stomach of an animal and moveable between a first orientation and a second orientation, comprising,
a sensor, and
an active data transmitter; and
a base station, comprising,
a data receiver, said data receiver having a first antenna with a first antenna orientation and a second antenna with a second antenna orientation, wherein said first antenna orientation substantially corresponds to said first bolus orientation and wherein said second antenna orientation substantially corresponds to said second bolus orientation.

2. The apparatus of claim 1, wherein said sensor comprises an accelerometer, said accelerometer to measure movement characteristics of said animal and where said movement characteristics comprise one selected from the group consisting of movement frequency, movement distance, movement rate, acceleration, and derivative of acceleration.

3. The apparatus of claim 2, wherein said accelerometer is a 3-axis accelerometer and wherein said movement characteristics are obtained by calculating the derivative of a vector magnitude obtained from said 3-axis accelerometer.

4. The apparatus of claim 1, wherein said first antenna orientation is substantially vertical and wherein said second antenna orientation is substantially horizontal.

5. The apparatus of claim 2, said sensor comprises one selected from the group consisting of a temperature sensor, a stomach pH sensor, a blood pH sensor, a global positioning system receiver, a heart rate monitor, a respiration monitor, and a rumen contraction sensor.

6. The apparatus of claim 4, wherein said active data transmitter is configured to transmit a message comprising data corresponding to said sensor.

7. The apparatus of claim 6, wherein said message comprises a media access control (MAC) value corresponding to said active data transmitter.

8. The apparatus of claim 6, wherein said message comprises an animal identifier value corresponding to said animal.

9. The apparatus of claim 6, wherein said message comprises a bolus identifier value corresponding to said bolus.

10. The apparatus of claim 3, wherein said bolus further comprises a memory unit communicatively coupled to said sensor.

11. The apparatus of claim 10, wherein said bolus further comprises a processor communicatively coupled to said memory unit and to said sensor, and wherein said memory unit comprises machine readable instructions to be executed by said processor.

12. The apparatus of claim 11, wherein said processor is configured to poll said sensor at a polling frequency, and wherein said polling frequency is determined by said machine readable instructions.

13. The apparatus of claim 11, wherein said processor modifies said polling frequency responsive to a measurement of said sensor.

14. The apparatus of claim 11, wherein said processor is configured to cause said active data transmitter to transmit a message comprising data corresponding to a measurement of said sensor.

15. The apparatus of claim 11, wherein said polling comprises said processor obtaining measurement data from said sensor and storing said measurement data on said memory unit, and wherein said processor causes said active data transmitter to transmit a message comprising data corresponding to said measurement data stored on said memory unit at a transmission interval.

16. The apparatus of claim 11, wherein said bolus further comprises a bolus data receiver in wireless communication with said base station.

17. The apparatus of claim 16, wherein said bolus data receiver is configured to receive machine readable instructions to be executed by said processor, and wherein said received instructions are stored on said memory unit.

18. The apparatus of claim 16, wherein said bolus data receiver is configured to receive an animal identifier value, and wherein said animal identifier value is stored on said memory unit.

19. The apparatus of claim 16, wherein said bolus data receiver is configured to receive calibration data corresponding to said sensor, and wherein said calibration data is stored on said memory unit.

20. The apparatus of claim 6, wherein said base station is configured to receive said message transmitted from said active data transmitter.

21. The apparatus of claim 20, wherein said base station is configured to detect a health condition responsive to said message.

22. The apparatus of claim 21, wherein said base station is configured to issue an alert to an animal manager corresponding to said detected health condition.

23. The apparatus of claim 22, wherein said base station is communicatively coupled to a local area network and wherein said alert corresponding to said health condition comprises one selected from the group consisting of an email message, an instant message, and a short message service message.

24. The apparatus of claim 22, wherein said base station is communicatively coupled to a cellular telephone network and wherein said alert corresponding to said health condition comprises one selected from the group consisting of an audio message, a text message, a short message service message.

25. The apparatus of claim 20, wherein said base station further comprises a data transmitter, and wherein said base station data transmitter is configured to transmit a message to said bolus responsive to receiving said bolus message.

26. The apparatus of claim 25, wherein said response message from said base station comprises machine readable instructions corresponding to one selected from the group consisting of a polling frequency of said bolus, a sampling frequency of one of said one or more sensors, a sampling duration of one of said one or more sensors, an operational mode of said bolus, and a transmission interval of said bolus.

27. The apparatus of claim 1, wherein said first orientation of said first base station antenna is substantially orthogonal to said second orientation of said second base station antenna.

28. The apparatus of claim 27, wherein a first wireless signal is received on said first base station antenna and a second wireless signal is received on said second base station antenna, and wherein said base station is configured to combine into a single signal said first signal received in said first antenna and said second signal received on said second antenna.

29. The apparatus of claim 25, wherein said base station data transmitter comprises a first base station transmitter antenna with a first orientation and a second base station transmitter antenna with a second orientation, and wherein said first base station transmitter antenna orientation substantially corresponds to said first bolus orientation and wherein said second base station transmitter antenna orientation substantially corresponds to said second bolus orientation.

30. The apparatus of claim 29, wherein said first base station transmitter antenna orientation is substantially orthogonal to said second base station transmitter antenna orientation.

31. A method of monitoring an animal, comprising:
receiving a first radio frequency signal on a first antenna having a first orientation;
receiving a second radio frequency signal on a second antenna having a second orientation; and
combining said first signal and said second signal into a single received signal, said received signal comprising an animal characteristics message.

32. The method of claim 31, wherein said first orientation of said first antenna substantially corresponds to a possible orientation of a bolus disposed within an animal.

33. The method of claim 32, wherein said second orientation of said second antenna substantially corresponds to a possible orientation of a bolus disposed within an animal.

34. The method of claim 33, wherein said first orientation of said first antenna is substantially orthogonal to said second orientation of said second antenna.

35. The method of claim 34, wherein said first orientation of said first antenna is substantially vertical and wherein said second orientation of said second antenna is substantially horizontal.

36. The method of claim 31, further comprising transmitting a signal from a bolus, said signal comprising an animal characteristics message.

37. The method of claim 36, further comprising disposing said bolus within the stomach of a ruminant animal.

38. The method of claim 37, wherein said bolus is configured to be maintained within the stomach of a ruminant animal in a first orientation or a second orientation and wherein said first antenna orientation substantially corresponds to said first bolus orientation and wherein said second antenna orientation substantially corresponds to said second bolus orientation.

39. The method of claim 36, wherein said animal characteristics message comprises one selected from the group consisting of a media access control value, an animal identifier value, and a bolus identifier value.

40. An apparatus for wirelessly monitoring an animal, comprising:
an ingestible bolus configured to be disposed within a stomach of a ruminant animal and movable between a plurality of orientations, comprising,
a sensor,
a memory unit communicatively coupled to said sensor,
a processor communicatively coupled to said memory unit and said sensor,
an active wireless data transmitter,
an active wireless data receiver, and
means for powering said sensor, said memory unit, said processor, said active data transmitter, and said active data receiver; and
a base station, comprising,
a wireless data receiver having a plurality of receiver antennae, wherein each of said plurality of receiver antennae has a different orientation corresponding to one of said plurality of bolus orientations, and
an wireless data transmitter having a plurality of transmitter antennae, wherein each of said plurality of transmitter antennae has a different orientation corresponding to one of said plurality of bolus orientations.
